(19) United States
(12) Patent Application Publication
(10) Pub. No.: US 2012/0220972 A1
(43) Pub. Date: Aug. 30, 2012

(54) ABSORBENT ARTICLE

(75) Inventors: Koji Kawamura, Kagawa (JP); Noriyuki Kurita, Kagawa (JP)

(73) Assignee: UNI-CHARM CORPORATION, Ehime (JP)

(21) Appl. No.: 13/508,130

(22) PCT Filed: Nov. 10, 2010
(86) PCT No.: PCT/JP2010/069973

§ 371 Date: May 4, 2012

(30) Foreign Application Priority Data
Nov. 13, 2009 (JP) 2009-260483

(51) Int. Cl. A61F 13/512

(52) U.S. Cl. 604/383

(57) ABSTRACT

An absorbent article adapted to accelerate an absorption rate of the absorbent member, to enlarge an absorption area in the absorbent member and to improve an absorption capacity of the absorbent member. A urine absorption pad includes a topsheet, a backsheet, a first layer element and a second layer element. The first layer element is formed with a slot-like first through hole extending nearly vertically in a thickness direction from its upper surface to its lower surface. The second layer element is formed with a slot-like second through hole extending nearly vertically in the thickness direction from its upper surface to its lower surface and overlapping with the first through hole. An opening area of the first through hole is larger than that of the second through hole and, in an overlapping state of the first through hole and the second through hole, front and rear ends and opposite side edges of the second through hole extend inward from front and rear ends and opposite side edges of the first through hole so as to define a stepped region between the first through hole and the second through hole.

### TECHNICAL FIELD

The present disclosure relates to absorbent articles and, more particularly, to absorbent articles such as urine absorption pads, disposable diapers, toilet-training pants, incontinent briefs and the like.

### BACKGROUND

Absorbent articles including a topsheet, a backsheet and an absorbent member interposed between these sheets are known. For example, PTL 1 discloses an absorbent article includes an absorbent member formed with through holes so that bodily fluids such as urine may pass downward the through holes. In such absorbent article, urine discharged on the topsheet may be absorbed on the topsheet side of the absorbent member and urine having passed downward the through holes to the backsheet side may be absorbed also on the backsheet side of the absorbent member. In this way, it is possible for such absorbent article to absorb urine over a wider range than the case in which urine discharged on the topsheet is absorbed by the absorbent member only on one side of the absorbent member.

### CITATION LIST

### Patent Literature

PTL 1: JP 2008-284190 A

### SUMMARY OF INVENTION

### Technical Problem

In the above-mentioned through holes, an opening area on the side of the topsheet is substantially the same as an opening area on the side of the backsheet and a cross-sectional surface of the through holes is substantially perpendicular to the absorbent member. Bodily fluids may be absorbed not only on the topsheet side but also on the backsheet side of the absorbent member. However, it cannot be found from the disclosure of PTL 1 that the cross-sectional surface also may be used to absorb bodily fluids.

The present invention provides an absorbent article adapted to accelerate an absorption rate of the absorbent member, to enlarge an absorption area in the absorbent member and thereby to improve an absorption capacity of the absorbent member.

### Solution to Problem

According to this invention, there is provided an absorbent article having a longitudinal direction and a transverse direction and including a topsheet lying on a side facing the wearer's body, a backsheet lying on a side opposite to the side facing the wearer's body and an absorbent member interposed between the topsheet and the backsheet wherein the absorbent member is formed with a through hole extending through the absorbent member from an upper surface thereof lying on the side of the topsheet to a lower surface thereof lying on the side of the backsheet.

In the above-mentioned absorbent article, the through hole has an upper opening on the upper surface, a lower opening on the lower surface and a peripheral wall connecting the upper opening to the lower opening, and at least one of front and rear ends in the longitudinal direction of the upper opening is not aligned in a thickness direction with front and rear ends in the longitudinal direction of the lower opening and the peripheral wall extends down- and inward from the front and rear ends in the longitudinal direction of the upper opening to the front and rear ends of the lower opening.

According to one embodiment of this invention, an opening area of the upper opening is larger than that of the lower opening and a dimension in the longitudinal direction of the upper opening is larger than that of the lower opening.

According to another embodiment of this invention, one of the front and rear ends of the lower opening extends outward in the longitudinal direction beyond the corresponding end of the upper opening.

According to yet another embodiment of this invention, the through hole has a dimension in the longitudinal direction larger than a dimension thereof in the transverse direction.

According to still another embodiment of this invention, the peripheral wall is formed with a stepped region created by a thickness dimension difference of the peripheral wall and extending in a circumferential direction.

According to further another embodiment of this invention, upper and lower surfaces of the absorbent member are covered with liquid diffusion sheets, respectively.

According to an alternative embodiment of this invention, the absorbent member includes a plurality of layer elements stacked in the thickness direction and the liquid diffusion sheet is interposed between each pair of the adjacent layer elements.

According to another alternative embodiment of this invention, the absorbent member is formed outboard of the through hole as viewed in the transverse direction with compressed grooves extending in the longitudinal direction.

According to yet another alternative embodiment of this invention, between the lower surface of the absorbent member and the backsheet, there are provided two or more bonding means spaced apart from each other in the transverse direction and extending in the longitudinal direction.

### Advantageous Effects of Invention

According to this invention, the through hole formed in the absorbent member has the peripheral wall extends down- and inward from the front and rear ends of the upper opening to the front and rear ends of the lower opening so that an area of the peripheral wall exposed to the upper opening may be enlarged in comparison with the case in which the peripheral wall vertically extends between the upper and lower openings. Correspondingly, it is possible to enlarge an absorption area in the absorbent member, to accelerate an absorption rate of the absorbent member, and to improve an absorption capacity of the absorbent member.

In addition, the peripheral wall extending down- and inward from the front and rear ends of the upper opening to the front and rear ends of the lower opening has a thickness smaller than the remaining region and advantageously provides a relatively high flexibility along the fold lines extending in the longitudinal direction so that, even when the absorbent article is folded along these fold lines, such a high flexibility functions to prevent the opposite side edges of the lower opening from coming in contact with each other and closing the opening.

### BRIEF DESCRIPTION OF DRAWINGS

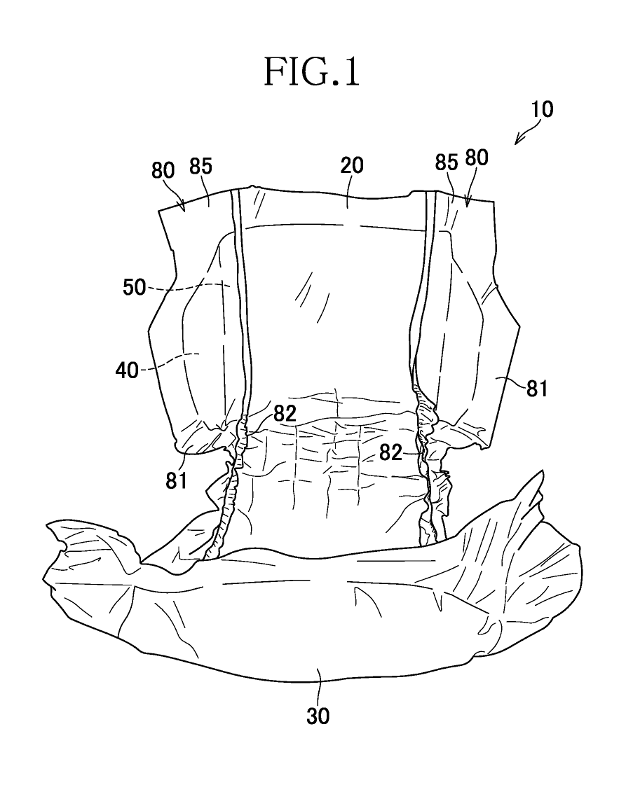

{FIG. 1} FIG. 1 is a perspective view of a urine absorption pad as one example of the absorbent article.

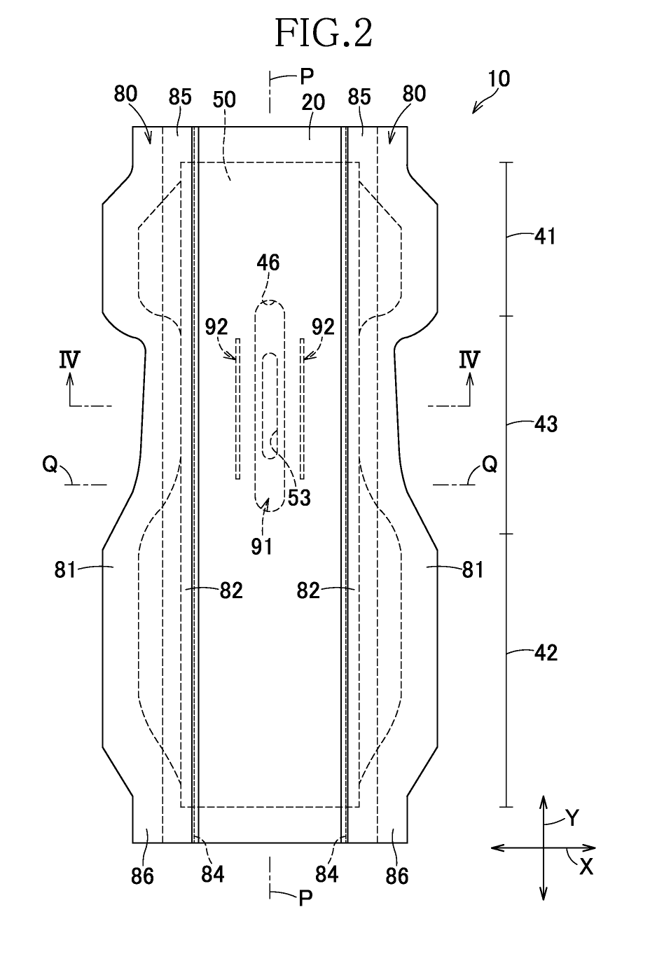

{FIG. 2} FIG. 2 is a developed plan view of a first embodiment.

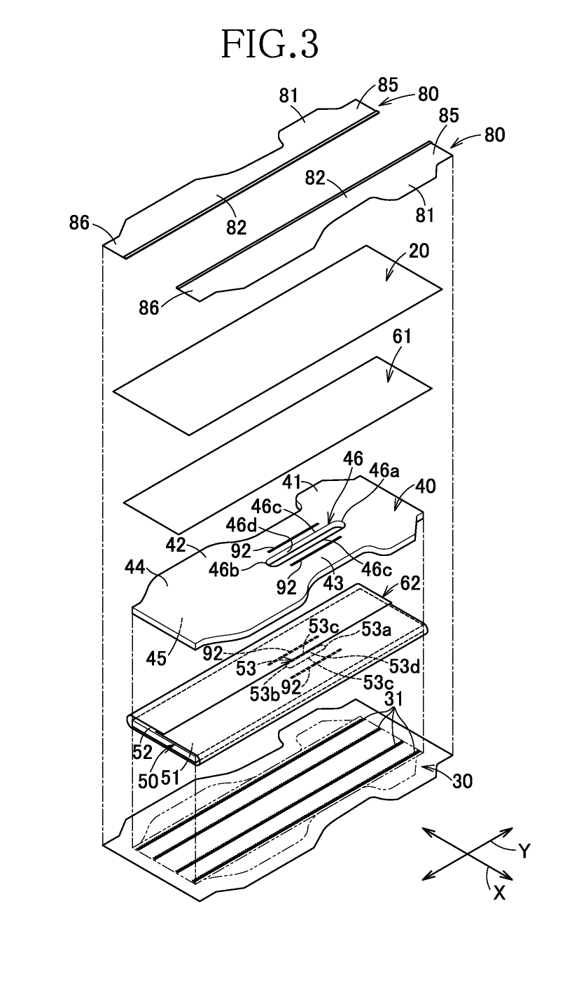

{FIG. 3} FIG. 3 is an exploded view of FIG. 2.

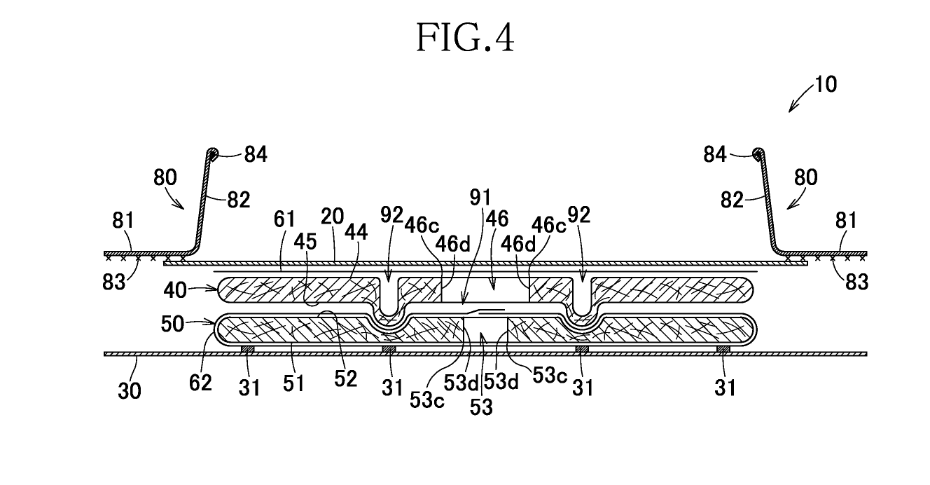

{FIG. 4} FIG. 4 is a sectional view taken along line IV-IV in FIG. 2.

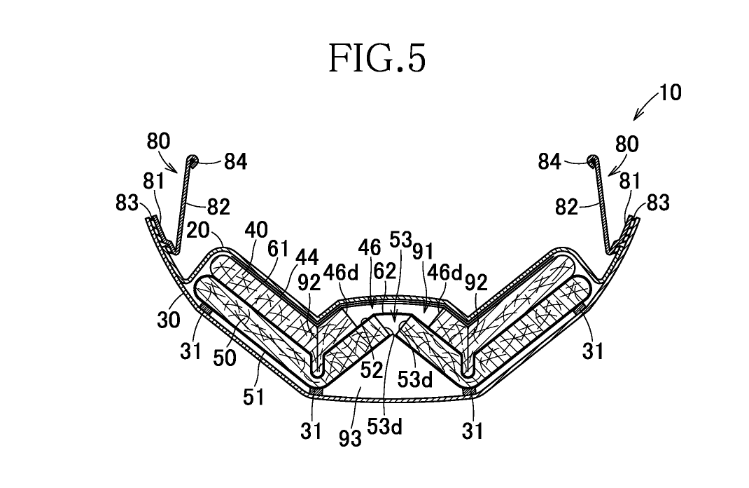

{FIG. 5} FIG. 5 is a diagram illustrating FIG. 4 in a state put on the wearer's body.

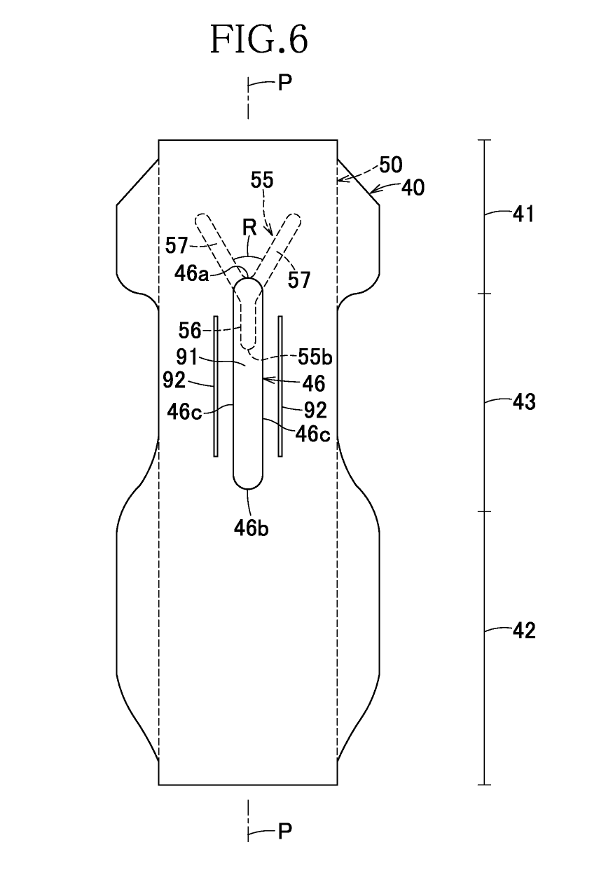

{FIG. 6} FIG. 6 is a plan view of the absorbent article according to a second embodiment.

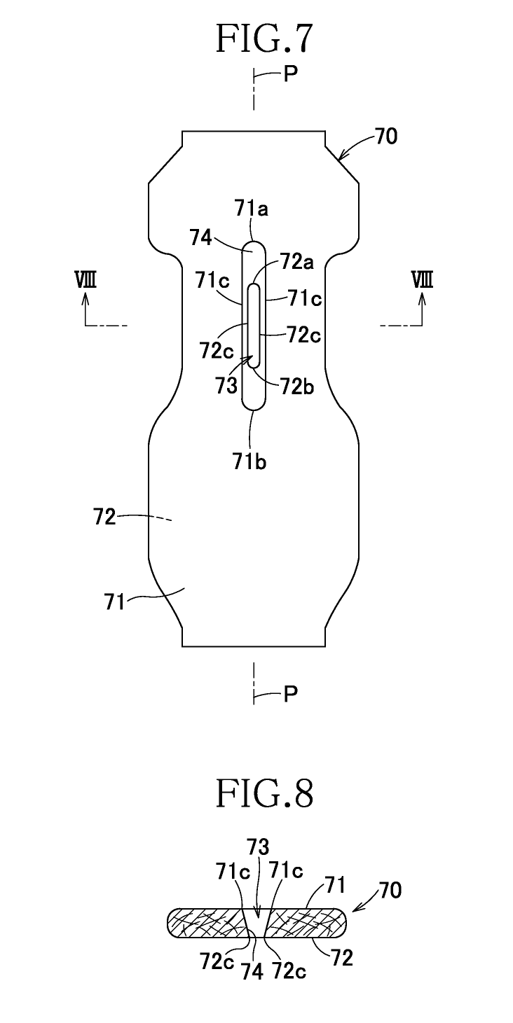

{FIG. 7} FIG. 7 is a plan view of the absorbent article according to a third embodiment.

{FIG. 8} FIG. 8 is a sectional view taken along line VIII-VIII in FIG. 7.

### DESCRIPTION OF EMBODIMENTS

### First Embodiment

FIGS. 1 through 5 illustrate a first embodiment of this invention and, referring to FIGS. 1 through 5, the absorbent article according to this invention will be described hereunder on the basis of a urine absorption pad as one example thereof.

FIG. 1 is a perspective view of a urine absorption pad 10, FIG. 2 is a developed plan view of the urine absorption pad 10 as viewed from the side of the wearer's body wherein elastic members are stretched against contractile force thereof to keep the urine absorption pad 10 in a flat state, FIG. 3 is an exploded view of FIG. 2, FIG. 4 is a sectional view taken along line IV-IV in FIG. 2 and FIG. 5 is a sectional view similar to FIG. 4, illustrating the sectional view when the urine absorption pad 10 is put on the wearer's body. In this regard, the respective sectional views are schematic diagrams in which respective sheets are illustrated so as to be spaced apart from each other in order to clarify the manner in which respective sheets are stacked in FIG. 4, elastic members 84 for leakage-barrier cuffs 80 are illustrated in a contraction state thereof. The urine absorption pad 10 includes an imaginary longitudinal center line P-P bisecting a dimension in a transverse direction X and an imaginary transverse center line Q-Q bisecting a dimension in a longitudinal direction Y and an outer shape of the urine absorption pad 10 is substantially symmetric about the imaginary longitudinal center line P-P.

The urine absorption pad 10 has the longitudinal direction Y and the transverse direction X and includes a topsheet 20 lying on the side of the wearer's body, a backsheet 30 lying on the side opposite to the topsheet 20, i.e. , on the side of the wearer's garment and an absorbent member interposed between the topsheet 20 and the backsheet 30. The topsheet 20 is liquid-permeable and may be formed, for example, of a polypropylene spun bonded fiber nonwoven fabric having a mass per unit area of about 20 g/m² and hydrophilized. The backsheet 30 is liquid-impermeable and may be formed, for example, of a polyethylene film having a mass per unit area of about 23.5 g/m² and water repellent finished.

The absorbent member includes a first layer element 40 stacked in the thickness direction and lying on the side of the topsheet 20 and a second layer element 50 stacked in the thickness direction and lying on the side of the backsheet 30. The first and second layer elements 40, 50 respectively include liquid-absorbent core material formed of absorbent pulp and/or superabsorbent polymer particles.

A first liquid diffusion sheet 61 is interposed between the first layer element 40 and the topsheet 20 and upper and lower surfaces of the second layer element 50 are wrapped with a second liquid diffusion sheet 62. As the first and second liquid diffusion sheets 61, 62, for example, tissue paper or a fibrous nonwoven fabric having a mass per unit area of about 15 g/m² may be used.

The topsheet 20 is provided on the side of the wearer's body with a pair of leakage-barrier cuffs 80 symmetrically about the imaginary longitudinal center line P-P so as to be spaced apart from each other in the transverse direction X. Respective outer edges 81 in the transverse direction X of the leakage-barrier cuffs 80 are bonded to the topsheet 20 and the backsheet 30 by bonding means 83 such as hot melt adhesives. Respective inner side edges 82 are not bonded to the topsheet 20 so that the inner side edges 82 may be spaced apart from the topsheet 20 (See FIG. 4). The respective inner side edges 82 form sleeves and elastic members 84 are attached within these sleeves.

The elastic members 84 extend in the longitudinal direction Y and are attached under tension and in a contractible manner. Front and rear ends 85, 86 of the respective leakage-barrier cuffs 80 are bonded to the topsheet 20 and the backsheet 30 by bonding means such as hot melt adhesives (not shown). With the urine absorption pad 10 put on the wearer's body, the inner side edges 82 are spaced upward from the topsheet 20 under contraction of the elastic members 84 and thereby may prevent bodily waste such as urine from leaking out beyond the opposite side edges of the urine absorption pad 10. As the leakage-barrier cuffs 80, for example, a spun bonded/melt blown/spun bonded (SMS) nonwoven fabric made of polypropylene fibers having a mass per unit area of about 15 g/m² may be used.

The first layer element 40 has front and rear sections 41, 42 spaced apart from each other in the longitudinal direction Y and a constricted section 43 extending between these front and rear sections 41, 42 and bent or curved in the vicinity of the imaginary transverse center line Q-Q so that a dimension thereof in the transverse direction X is reduced. The first layer element 40 and the backsheet 30 are substantially similar in outer shapes thereof but the backsheet 30 is sized to be larger than the first layer element 40. The first layer element 40 has an upper surface 44 facing the topsheet 20 and a lower surface 45 facing the second layer element 50 and the first layer element 40 is formed with a slot-like first through hole 46 extending nearly vertically in the thickness direction from the upper surface 44 to the lower surface 45.

The first through hole 46 lies at least in the constricted section 43 and, in this embodiment, the first through hole 46 extends slightly beyond the constricted section 43 to the front and rear sections 41, 42. The first through hole 46 extends in the longitudinal direction Y so as to overlap the imaginary longitudinal center line P-P. In this embodiment, a dimension in the longitudinal direction Y of the first through hole 46 is about 180 mm and a dimension in the transverse direction X is about 25 mm. The first through hole 46 defines, on the upper surface 44, the upper opening including front and rear ends 46a, 46b and opposite side edges 46c and defines, in the thickness direction, a peripheral wall 46d.

The second layer element 50 has a substantially rectangular outer shape and has a dimension in the transverse direction X substantially equal to that of the constricted section 43 of the first layer element 40. The second layer element 50 has a lower surface 51 facing the backsheet 30 and an upper surface 52 facing the first layer element 40 and the second layer element 50 is formed with a slot-like second through hole 53 extending nearly vertically in the thickness direction from the upper surface 52 to the lower surface 51. The second through hole 53 extends in the longitudinal direction Y so as to overlap the imaginary longitudinal center line P-P and also to overlap the first through hole 46. The second through hole 53 defines, on the lower surface 51, the lower opening including front and rear ends 53a, 53b and opposite side edges 53c and defines, in the thickness direction, a peripheral wall 53d.

In this embodiment, a dimension in the longitudinal direction Y of the second through hole 53 is about 90 mm and a dimension in the transverse direction X thereof is about 12 mm. With such dimensioning, the first through hole 46 has an opening area larger than that of the second through hole 53 and, when the first layer element 40 is lapped over the second layer element 50, the opening of the second through hole 53 is exposed to the inside of the opening of the first through hole 46. In other words, the front and rear ends 53a, 53b and the opposite side edges 53c if the second through hole 53 extend inward from the front and rear ends 46a, 46b and the opposite side edges 46c of the first through hole 46 to forma stepped region 91 between the first through hole 46 and the second through hole 53.

The stepped region 91 is defined by the peripheral wall 46d of the first through hole 46, the upper surface of the second layer element 50 and the peripheral wall 53d of the second through hole 53. Specifically, the stepped region 91 includes a portion in which the first layer element 40 and the second layer element 50 lapped over each other and a portion defined by the second layer element 50 only and a thickness difference resulting from such partial lapping defines the stepped region 91. The stepped region 91 formed in this manner makes it possible to improve the absorption capacity for bodily fluids in comparison with the case in which the peripheral wall 46d and the peripheral wall 53d are aligned with each other in the thickness direction and extend almost vertically. More specifically, while the effect of bodily fluid-absorption obtained in the through holes is limited to the continuous peripheral wall of the first through hole 46 and the second through hole 53 if the opening area of the first through hole 46 is equal to the opening area of the second through hole 53, according to this embodiment, in contrast, bodily fluids may be absorbed not only along the respective peripheral walls 46d, 53d of the first through hole 46 and the second through hole 53 but also the upper surface 52 of the second layer element 50. In this way, it is possible to accelerate the absorption rate and thereby to improve the absorption capacity.

The above-mentioned first layer element 40 and second layer element 50 are respectively provided outboard of the first through hole 46 and the second through hole 53 as viewed in the transverse direction X with compressed grooves 92. The compressed grooves 92 are formed by compressing from the upper surface 44 toward the lower surface 51 under heating the first layer element 40, the second layer element 50 and the liquid diffusion sheets 61, 62 stacked on each other. The compressed grooves 92 formed in this manner serve to tighten a stacking of the first layer element 40 and the second layer element 50 and, at the same time, increase a density of the first and second layer elements in the vicinity of the respective compressed grooves 92 so that bodily fluids such as urine may be smoothly guided from the upper surface 44 toward the lower surface 51. In addition, with the urine absorption pad 10 put on the wearer's body, the urine absorption pad 10 is easily folded along the compressed grooves 92. In this embodiment, a dimension in the longitudinal direction Y of the first through hole 46 is larger than a dimension in the longitudinal direction Y of the compressed grooves 92.

The second layer element 50 is bonded to the backsheet 30 by bonding means 31 such as hot melt adhesives by the intermediary of the second liquid diffusion sheet 62. Two or more these bonding means 31 extend in the longitudinal direction Y and are spaced apart from each other in the transverse direction X.

The urine absorption pad 10 as has been described above is preferably put on the wearer's body so that the first and second through holes 46, 53 may be aligned with the wearer's excretory. The urine absorption pad 10 may be used in combination with a separately prepared diaper, for example, by attaching the urine absorption pad 10 to the inside of the diaper. It is also possible to use the urine absorption pad 10 by attaching it to the wearer's underwear or cover. Urine discharged on the urine absorption pad 10 put on the wearer's body in such manners permeates the topsheet 20, then is diffused by the first liquid diffusion sheet 61 in the longitudinal direction Y as well as in the transverse direction X and absorbed by the first layer element 40 and partially flows into the first through hole 46. Urine having flown into the first through hole 46 may be absorbed also through the stepped region 91 as has previously been described.

The second liquid diffusion sheet 62 is interposed between the first layer element 40 and the second layer element 50 and discharged urine is transferred from the first layer element 40 to the second layer element 50 along the second liquid diffusion sheet 62 and absorbed by the first layer element 40 or the second layer element 50. Urine having flown down along the first through hole 46 and the second through hole 53 is transferred to the boundary between the second layer element 50 and the backsheet 30 and may be absorbed by the second layer element 50 from the lower surface 51. The second liquid diffusion sheet 62 is interposed between the second layered region 50 and the backsheet 30 and therefore urine having attained this level is diffused along the second liquid diffusion sheet 62 in the longitudinal direction Y as well as in the transverse direction X and simultaneously absorbed by the second layer element 50.

As has been described above, interposition of the first and second liquid diffusion sheets 61, 62 makes it possible to utilize the liquid-absorbent core material over a wide range. In other words, it is possible to restrict the problem that the discharged urine might be locally absorbed by the absorbent member and, in consequence, the absorption rate might be lowered and the absorption region might be limited.

FIG. 5 is a diagram corresponds to FIG. 4 except that FIG. 5 illustrates the sectional diagram of FIG. 4 when the urine absorption pad 10 is put on the wearer's body. The urine absorption pad 10 is preferably put on the wearer's body so that the front section 41 of the first layer element 40 lies in a front waist side so as to cover the wearer's external genital and the rear section 42 lies in the wearer's rear waist side so as to cover the wearer's buttocks. With the urine absorption pad 10 put on the wearer's body in such a manner, the constricted section 43 is squeezed by the wearer's inguinal regions or thighs from outside toward inside in the transverse direction X. When the urine absorption pad 10 is squeezed in this manner, the cross-section thereof is folded in W-shape as illustrated in FIG. 5.

In the urine absorption pad 10, a stiffness difference between the compressed grooves 92 and the first and second through holes 46, 53 and the remaining region facilitates the fold lines extending in parallel to the imaginary longitudinal center line P-P to be formed. The midsection in the transverse direction X including the first and second through holes 46, 53, the pad is folded closer to the wearer's body and the compressed grooves 92 are folded so as to be spaced apart from the wearer's body. In this way, the cross-section of the pad 10 is folded in W-shape and further facilitates discharged urine to flow into the first and second through holes 46, 53.

The bonding means 31 are provided between the second layer element 50 and the backsheet 30 so as to be spaced apart from each other in the transverse direction X so that, between each pair of the bonding means 31, the second layer element 50 is spaced apart from the backsheet 30 to define a void space 93. These void spaces 93 may temporarily retain discharged urine. Even when superabsorbent polymer particles or the like having a relatively low initial absorption rate is used for the liquid-absorbent core material, such void spaces 93 ensure discharged urine to be completely absorbed.

In the above-mentioned urine absorption pad 10, the stepped regions 91 define portions thinner than the remaining portion between the front and rear ends 46a, 46b of the first through hole 46 and the front and rear ends 53a, 53b of the second through hole 53. These thinner portions have a stiffness lower than that of the remaining portion and provide a relatively high flexibility facilitating the pad to be folded the aforementioned fold lines. Particularly in the state as illustrated in FIG. 5, such a high flexibility may prevent the opposite side edges 53c from coming in contact with each other and thereby closing this through hole 53. If the through hole 53 is closed, discharged urine may not flow downward to the lower surface 51 of the second layer element 50 and absorption of urine from the lower surface may not be expected. In contrast, the embodiment according to this invention may effectively overcome such a problem. In this regard, in order that the absorbent member may have an appropriate flexibility, the liquid-absorbent core material is preferably formed of pulp or the like having a relatively long fiber length.

In the constitution described above, the first and second layer elements 40, 50 may be formed by layering liquid-absorbent core material such as pulp fibers in the thickness direction. In this case, the first and second through holes 46, 53 may be formed by layering the core material so that none of the core material is stacked in regions corresponding to the first and second through holes 46, 53. Alternatively, it is also possible to layer the core material over respective whole areas corresponding to the first and second layer elements 40, 50 and thereafter to hollow out the first and second through holes 46, 53 from the respective layer elements 40, 50.

While the first and second liquid diffusion sheets 61, 62 are spaced apart from the respective peripheral walls 46d, 53d of the first and second through holes 46, 53 according to this embodiment, it is also possible to put these sheets 61, 62 in close contact with the respective peripheral walls 46d, 53d. By putting the first and second liquid diffusion sheets 61, 62 in close contact with the respective peripheral walls 46d, 53d, it is possible to improve urine absorption efficiency from the peripheral walls.

According to this embodiment, the second through hole 53 is positioned roughly in the center of the first through hole 46 so that the front and rear ends 53a, 53b and the opposite side edges 53c of the second through hole 53 extend inward from the front and rear ends 46a, 46b and the opposite side edges 46c of the first through hole 46 so that the stepped region 91 is formed fully around the peripheral walls 46d, 53d in the circumferential direction. In this regard, however, it is possible to form the stepped region 91 on any one of the front and rear ends 46a, 46b to form along respective parts of the peripheral walls 46d and 53d in the circumferential direction. Specifically, an alternative arrangement is also possible such that the first and second through holes 46, 53 have the same dimension in the transverse direction X and the stepped region 91 is not formed in the transverse direction X. Furthermore, an alternative arrangement is also possible such that the second through hole 53 is eccentrically-located in the longitudinal direction Y of the first through hole 46 and the stepped region 91 is formed only one of the front and rear ends 46a, 46b. In addition, it is not essential that the second through hole 53 is positioned inside the first through hole 46 and one of the front and rear ends 53a, 53b may extend outward beyond the first through hole 46.

According to this embodiment, the first through hole 46 extends from the constricted section 43 extends to the front and rear sections 41, 42 and are dimensioned to be longer than the compressed grooves 92 in the longitudinal direction Y. The dimension in the longitudinal direction Y of the first and second through holes 46, 53 may be enlarged to enlarge the opening area on the upper surface 44 and thereby to absorb urine quickly, even if this amount is relatively large. Furthermore, the first and second layer elements 40, 50 are adequately long in the longitudinal direction Y and the first and second through holes 46, 53 also may be dimensioned adequately long in the longitudinal direction Y to utilize the liquid-absorbent core material of these first and second layer elements 40, 50 efficiently.

While the absorbent member has been described to include the first layer element 40 and the second layer element 50, this invention is not limited to such constitution and it is possible for the absorbent member to include three or more layer elements. In such a case, opening areas of the respective through holes may be dimensioned to be stepwise or continuously reduced from the uppermost layer element to the lowermost layer element. While the first and second through holes 46, 53 are formed in front of the imaginary transverse center line Q-Q in this embodiment, this invention is not limited to such location of the through holes 46, 53 and the locations of these through holes 46, 53 may be appropriately selected. Particularly, both the dimensions and the locations of the first and second through holes may be selected depending on a whole length in the longitudinal direction Y of the urine absorption pad 10. The first and second through holes 46, 53 respectively have a dimension in the longitudinal direction Y larger than a dimension in the transverse direction X. Such dimensioning makes it possible to ensure an adequately large urine absorption region when the wearer is in a side lying posture. Specifically, urine flowing into the first and second through holes 46, 53 is diffused within these through holes and simultaneously permeates through a horizontal surface extending over a dimension corresponding to the dimension in the longitudinal direction Y. Therefore, the larger the dimension in the longitudinal direction, the larger the area of the permeation region.

### Second Embodiment

FIG. 6 is a plan view of the absorbent member according to a second embodiment. In this embodiment, a second through region 55 is characterized in that the second through hole 55 includes a trunk segment 56 extending on the imaginary longitudinal center line P-P and a pair of branch segments 57 extending forward in the longitudinal direction Y from the trunk segment 56 and diverging in the transverse direction X. The other constituents are similar to those in the first embodiment and will not be repetitively described hereunder.

The first layer element 40 and the first through hole 46 are similar to those in the first embodiment. the second through hole 55 formed in the second layer element 50 includes the trunk segment 56 extending in the longitudinal direction Y and a pair of the branch segments 57 extending from the trunk segment 56 and is a Y-shaped as a whole. The branch segments 57 diverge forward and are spaced apart from each other at an angle R of about 60°. Of such second through hole 55, the trunk segment 56 overlaps with the first through hole 46 and the branch segments 57 extend from the first through hole 46. In other words, the branch segments 57 lie outboard of the front end 46a of the first through hole 46 as viewed in the longitudinal direction Y.

In this embodiment, a stepped region 91 is formed between the rear end 46b of the first through hole 46 and a rear end 55b of the second through hole 55.

With the urine absorption pad 10 having the constitution as has been described above, the first and second layer elements 40, 50 are folded along the first and second through holes 46, 55 in the same manner as in the first embodiment. In the second through hole 55, the urine absorption pad 10 is folded along the trunk segment 56 and the branch segments 57. The branch segments 57 are located in the front waist side and therefore the urine absorption pad 10 can be put in surface-contact with the wearer's body particularly in the vicinity of the excretory. In this way, the urine absorption pad 10 may be put in close contact with the wearer's body over a sufficiently large area for urine absorption.

The second through hole 55 lying outboard of the front end 46a of the first through hole 46 in the longitudinal direction Y makes it possible to diffuse discharged urine correspondingly enlarged area before absorption without reducing the absorption capacity of the first through hole 46. This is for the reason that, while the opening area of the first through hole 46 is not changed, urine flowing into the first through hole 46 may be diffused farther via the trunk segment 56 and the branch segments 57 of the second through hole 55.

While the branch segments 57 extend forward from the trunk segment 56 in this embodiment, it is possible to form the branch segments so as to extend rearward. Dimensions of the trunk segment 56 and the branch segments 57 as well as the relative positions thereof to the first through hole 46 are not limited to this embodiment and may be appropriately varied.

### Third Embodiment

FIG. 7 is a plan view showing the absorbent member according to a third embodiment and FIG. 8 is a sectional view taken along line VIII-VIII in FIG. 7. This embodiment is characterized in that a single layered absorbent member 70 is adopted. Other constitutions are similar to the first embodiment and will not be repetitively described hereunder.

The absorbent member 70 includes an upper surface 71 lying on the side of the topsheet, a lower surface 72 lying on the side of the backsheet and a through hole 73 extending through these upper and lower surfaces 71, 72. As a result of formation of the through hole 73, the upper surface 71 is formed with an upper opening contoured by front and rear ends 71a, 71b and opposite side edges 71c. The lower surface 72 is formed with a lower opening contoured by front and rear ends 72a, 72b and opposite side edges 72c. In addition, a peripheral wall 74 is formed in the thickness direction.

The peripheral wall extends down- and obliquely inward from the upper surface 71 toward the lower surface 72 without forming a stepped region. The peripheral wall 74 obliquely extends so that the front and rear ends 72a, 72b of the lower surface 72 lie inside the front and rear ends 71a, 71b of the upper surface 71 and the opposite side edges 72c of the lower surface 72 lie inside the opposite side edges 71c of the upper surface 71. In other words, an opening area of the upper surface 71 is larger than an opening area of the lower surface 72 and the lower opening is formed inside the upper opening so that the peripheral wall 75 may be visually recognized from immediately above the upper opening.

With the constitution as has been described above, the area of the peripheral wall exposed to the upper opening may be enlarged in comparison with the case in which the peripheral 74 extends vertically in the thickness direction from the upper surface 71 to the lower surface 72. In this way, it is possible to improve the urine absorption capacity. The peripheral wall 74 serves also to provide an appropriate flexibility so that the through hole 73 may be smoothly folded. It is also possible to provide the upper surface 71 and the lower surface 72 of the absorbent member 70 with the liquid diffusion sheets, respectively.

### REFERENCE SIGNS LIST

10 urine absorption pad (absorbent article)
20 topsheet
30 backsheet
31 bonding means
40 first layer element
44 upper surface
46 first through hole
46a front end
46b rear end
46d peripheral wall
50 second layer element
51 lower surface
53 second through hole
53a front end
53b rear end
53d peripheral wall
55 second through hole
61 first liquid diffusion sheet
62 second liquid diffusion sheet
70 absorbent member
71 upper surface
72 lower surface
73 through hole
74 peripheral wall
91 stepped region
92 compressed groove
X transverse direction
Y longitudinal direction

1. An absorbent article having a longitudinal direction and a transverse direction, the article including:
a topsheet lying on a side facing the wearer's body;
a backsheet lying on a side opposite to the side facing the wearer's body; and
an absorbent member interposed between the topsheet and the backsheet
the absorbent member is formed with a through hole extending through the absorbent member from an upper surface thereof lying on the side of the topsheet to a lower surface thereof lying on the side of the backsheet,
the through hole has an upper opening on the upper surface, a lower opening on the lower surface and a peripheral wall connecting the upper opening to the lower opening; and
at least one of front and rear ends in the longitudinal direction of the upper opening is not aligned in a thickness direction with front and rear ends in the longitudinal direction of the lower opening and the peripheral wall extends down- and inward from the front and rear ends in the longitudinal direction of the upper opening to the front and rear ends of the lower opening.

2. The absorbent article defined by claim 1, wherein an opening area of the upper opening is larger than that of the lower opening and a dimension in the longitudinal direction of the upper opening is larger than that of the lower opening.

3. The absorbent article defined by claim 1, wherein one of the front and rear ends of the lower opening extends outward in the longitudinal direction beyond the corresponding end of the upper opening.

4. The absorbent article defined by claim 1, wherein the through hole has a dimension in the longitudinal direction larger than a dimension thereof in the transverse direction.

5. The absorbent article defined by claim 1, wherein the peripheral wall is formed with a stepped region created by a thickness dimension difference of the peripheral wall and extending in a circumferential direction.

6. The absorbent article defined by claim 1, wherein upper and lower surfaces of the absorbent member are covered with liquid diffusion sheets, respectively.

7. The absorbent article defined by claim 1, wherein the absorbent member includes a plurality of layer elements stacked in the thickness direction and the liquid diffusion sheet is interposed between each pair of the adjacent layer elements.

8. The absorbent article defined by claim 1, wherein the absorbent member is formed outboard of the through hole as viewed in the transverse direction with compressed grooves extending in the longitudinal direction.

9. The absorbent article defined by claim 1, wherein, between the lower surface of the absorbent member and the backsheet, there are provided two or more bonding means spaced apart from each other in the transverse direction and extending in the longitudinal direction.

10. The absorbent article defined by claim 2, wherein one of the front and rear ends of the lower opening extends outward in the longitudinal direction beyond the corresponding end of the upper opening.

11. The absorbent article defined by claim 2, wherein the through hole has a dimension in the longitudinal direction larger than a dimension thereof in the transverse direction.

12. The absorbent article defined by claim 3, wherein the through hole has a dimension in the longitudinal direction larger than a dimension thereof in the transverse direction.

13. The absorbent article defined by claim 10, wherein the through hole has a dimension in the longitudinal direction larger than a dimension thereof in the transverse direction.

14. The absorbent article defined by claim 2, wherein the peripheral wall is formed with a stepped region created by a thickness dimension difference of the peripheral wall and extending in a circumferential direction.

15. The absorbent article defined by claim 3, wherein the peripheral wall is formed with a stepped region created by a thickness dimension difference of the peripheral wall and extending in a circumferential direction.

16. The absorbent article defined by claim 4, wherein the peripheral wall is formed with a stepped region created by a thickness dimension difference of the peripheral wall and extending in a circumferential direction.

17. The absorbent article defined by claim 10, wherein the peripheral wall is formed with a stepped region created by a thickness dimension difference of the peripheral wall and extending in a circumferential direction.

18. The absorbent article defined by claim 11, wherein the peripheral wall is formed with a stepped region created by a thickness dimension difference of the peripheral wall and extending in a circumferential direction.

19. The absorbent article defined by claim 12, wherein the peripheral wall is formed with a stepped region created by a thickness dimension difference of the peripheral wall and extending in a circumferential direction.

20. The absorbent article defined by claim 13, wherein the peripheral wall is formed with a stepped region created by a thickness dimension difference of the peripheral wall and extending in a circumferential direction.
